(19) United States
(12) Patent Application Publication
(10) Pub. No.: US 2026/0060591 A1
(43) Pub. Date: Mar. 5, 2026

(54) ELECTROCARDIOGRAM DISPLAY METHOD

(71) Applicant: QT Medical, Inc., Taipei City (TW)

(72) Inventors: Chien-Lun TSENG, Taipei City (TW); Yu-Ting CHEN, Taipei City (TW)

(21) Appl. No.: 19/295,688

(22) Filed: Aug. 10, 2025

(30) Foreign Application Priority Data
Sep. 4, 2024 (TW) 113133543

(51) Int. Cl. A61B 5/339; A61B 5/358

(52) U.S. Cl. CPC A61B 5/339; A61B 5/358

(57) ABSTRACT

An electrocardiogram display method includes the following steps. First, the electrocardiogram is measured, the ST amplitudes of multiple electrocardiogram leads are then calculated based on the measured electrocardiogram, the measured electrocardiograms are classified according to regions of the heart, and bar charts of the ST amplitudes of the electrocardiogram leads are drawn according to the region classification.

### RELATED APPLICATIONS

This application claims priority to Taiwan Application Serial Number 113133543 filed Sep. 4, 2024, the disclosures of which are incorporated herein by reference in their entireties.

### BACKGROUND

### Technical Field

The present invention relates to an electrocardiogram display method. More particularly, the present invention relates to a rapid electrocardiogram display method.

### Description of Related Art

With the continuous advancement of technology and medical techniques, human lifespan has gradually increased. Currently, our country is gradually entering an aging society, with the proportion of the elderly population rising year by year, and heart disease has become one of the leading silent killers among the elderly.

Arrhythmia is a very common symptom among heart-related diseases, making the monitoring of heart-related diseases an important issue for the elderly. Currently, heart rate/ECG detection primarily uses ECG measurement equipment. Traditional methods involve attaching wet electrodes containing electrolytes to the chest, which act as a connection between the skin and the machine to measure heart rate/ECG.

With the advancement of technology, various ECG measurement devices have been developed. In addition to traditional wet electrodes, new disposable electrodes and specialized measurement electrodes designed for different body sizes have also matured.

However, after a patient's electrocardiogram is measured, professional medical personnel are required to interpret the ECG to correctly determine if the heart is abnormal. Traditional electrocardiograms do not allow medical personnel to quickly understand the location of heart abnormalities relative to the heart, leading to more time being needed to decide the treatment required for the patient.

Therefore, there is a need to provide a suitable ECG display method to accelerate medical personnel's judgment and treatment so as to improve patient survival and cure rates.

### SUMMARY

The summary of the present invention is intended to provide a simplified description of the disclosure to enable readers to have a basic understanding of the disclosure. The summary of the present invention is not a complete overview of the disclosure, and it is not intended to point out the importance of the embodiments/key elements of the present invention or define the scope of the invention.

One objective of the present invention is to provide an electrocardiogram display method for rapidly judging and treating heart abnormalities in patients.

To achieve these and other advantages and in accordance with the objective of the embodiments of the present invention, as the embodiment broadly describes herein, the embodiments of the present invention provides an electrocardiogram display method including the following steps. First, an electrocardiogram is measured, then, the ST amplitudes of a plurality of electrocardiogram leads are calculated according to the measured electrocardiogram, and the measured electrocardiograms are classified according to heart regions, and bar charts of the ST amplitudes of the electrocardiogram leads are drawn into predetermined distinct groups according to the classification.

In some embodiments, the electrocardiogram display method further includes marking abnormal threshold values for the electrocardiogram leads.

In some embodiments, the abnormal threshold values for the electrocardiogram leads in the electrocardiogram display method are drawn with red abnormal threshold lines.

In some embodiments, the electrocardiogram display method further includes comparing the ST amplitudes with corresponding abnormal threshold values of the abnormal threshold values, and issuing an alert when the ST amplitudes exceed the abnormal threshold values.

In some embodiments, the electrocardiogram display method further includes marking the actual ST amplitude values beside the bar charts of the ST amplitudes of the electrocardiogram leads.

In some embodiments, the electrocardiogram display method further includes drawing the names of the electrocardiogram leads in corresponding lead identification areas.

In some embodiments, the step of drawing bar charts of the ST amplitudes of the electrocardiogram leads into predetermined distinct groups includes drawing an anterior heart ECG group, drawing an inferior heart ECG group, and drawing a lateral heart ECG group.

In some embodiments, the step of drawing the anterior heart ECG group includes drawing a Lead V2 bar chart, drawing a Lead V3 bar chart, and drawing a Lead V4 bar chart.

In some embodiments, the step of drawing the inferior heart ECG group includes drawing a Lead II bar chart, drawing a Lead III bar chart, and drawing a Lead aVF bar chart.

In some embodiments, the step of drawing the lateral heart ECG group includes drawing a Lead I bar chart, drawing a Lead aVL bar chart, drawing a Lead V5 bar chart, and drawing a Lead V6 bar chart.

According to another aspect of the present invention, an electrocardiogram display method is provided, including the following steps. First, an electrocardiogram is measured, then, the ST amplitudes of a plurality of electrocardiogram leads are calculated according to the measured electrocardiogram, and the measured electrocardiograms are classified according to heart regions, and bar charts of the ST amplitudes of the electrocardiogram leads are drawn into an anterior heart ECG group, an inferior heart ECG group and a lateral heart ECG group.

In some embodiments, the electrocardiogram display method further includes marking abnormal threshold values for the electrocardiogram leads.

In some embodiments, the abnormal threshold values for the electrocardiogram leads in the electrocardiogram display method are drawn with red abnormal threshold lines.

In some embodiments, the electrocardiogram display method further includes comparing the ST amplitudes with corresponding abnormal threshold values of the abnormal threshold values, and issuing an alert when the ST amplitudes exceed the abnormal threshold values.

In some embodiments, the electrocardiogram display method further includes marking the actual ST amplitude values beside the bar charts of the ST amplitudes of the electrocardiogram leads.

In some embodiments, the electrocardiogram display method further includes drawing the names of the electrocardiogram leads in corresponding lead identification areas.

In some embodiments, the anterior heart ECG group includes a Lead V2 bar chart, a Lead V3 bar chart, and a Lead V4 bar chart.

In some embodiments, the inferior heart ECG group includes a Lead II bar chart, a Lead III bar chart, and a Lead aVF bar chart.

In some embodiments, the lateral heart ECG group includes a Lead I bar chart, a Lead aVL bar chart, a Lead V5 bar chart, and a Lead V6 bar chart.

Hence, the electrocardiogram display method disclosed in the present invention allows medical personnel to easily determine the site of heart damage based on different ECG groups, and the bar charts and abnormal threshold lines make the electrocardiogram convenient to recognize the magnitude of the abnormality. In combination with the actual ST amplitude values marked beside the corresponding bar charts, the electrocardiogram is more convenient for medical personnel to read abnormal values. Furthermore, the lead identification areas marked on the horizontal axis of the bar charts make the electrocardiogram easier for medical personnel to confirm the corresponding ECG leads of the bar charts. Therefore, the rapid electrocardiogram may effectively reduce the time for medical personnel to judge ECG abnormalities in emergencies, thereby improving the efficiency of diagnosis and treatment by medical personnel.

### BRIEF DESCRIPTION OF THE DRAWINGS

The disclosure may be more fully understood by reading the following detailed description of the embodiment, with reference made to the accompanying drawings as follows:

FIG. 1 is a schematic flowchart of an electrocardiogram display method according to an embodiment of the present invention;

FIG. 2 is a schematic diagram of an electrocardiogram; and

FIG. 3 is a schematic diagram of an electrocardiogram drawn by an electrocardiogram display method according to an embodiment of the present invention.

### DETAILED DESCRIPTION OF THE PREFERRED EMBODIMENT

The following is a detailed description of the embodiments in conjunction with the accompanying drawings, but the provided embodiments are not intended to limit the scope of the disclosure, and the description of the structure and operation is not utilized to limit the execution sequence thereof. The structure of the recombination of components and the resulting devices with equal functions are all within the scope of this disclosure. In addition, the drawings are for illustration purposes only, and are not drawn according to the original scale. For ease of understanding, the same reference numbers are utilized in the drawings and the description to refer to the same or like parts.

In addition, the terms utilized in the entire description and the scope of the patent application, unless otherwise specified, usually have the usual meaning of each term utilized in this field, in the content disclosed here and in the special content. Some terms utilized to describe the disclosure are discussed below or elsewhere in this specification to provide additional guidance to those skilled in the art in the disclosure.

In the implementation mode and the scope of the present application, unless the article is specifically limited in the context, “a” and “the” may generally refer to a single or pluralities. In the steps, the numbering is only utilized to conveniently describe the steps, rather than to limit the sequence and implementation.

Secondly, the words “comprising”, “including”, “having”, “containing” and the like utilized in the present application are all open language, meaning including but not limited thereto.

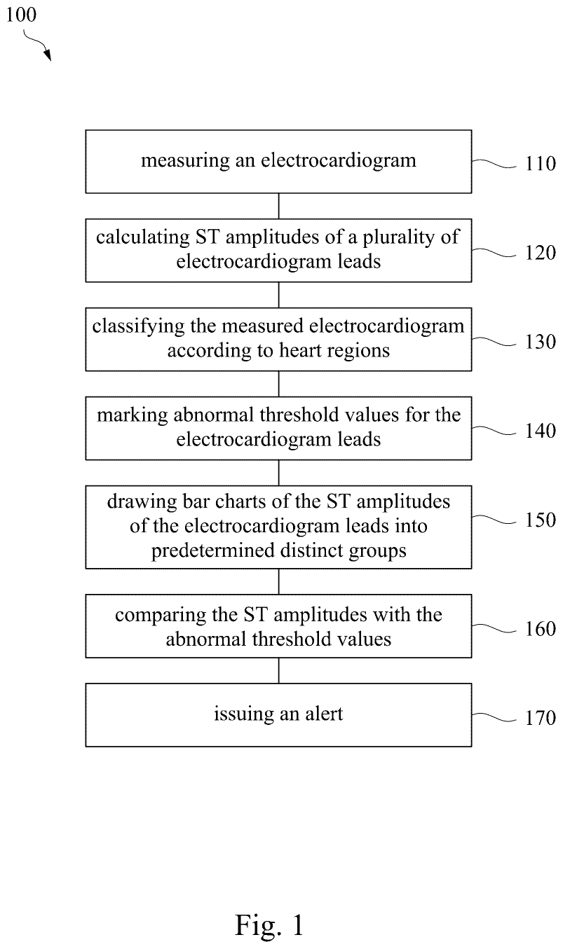

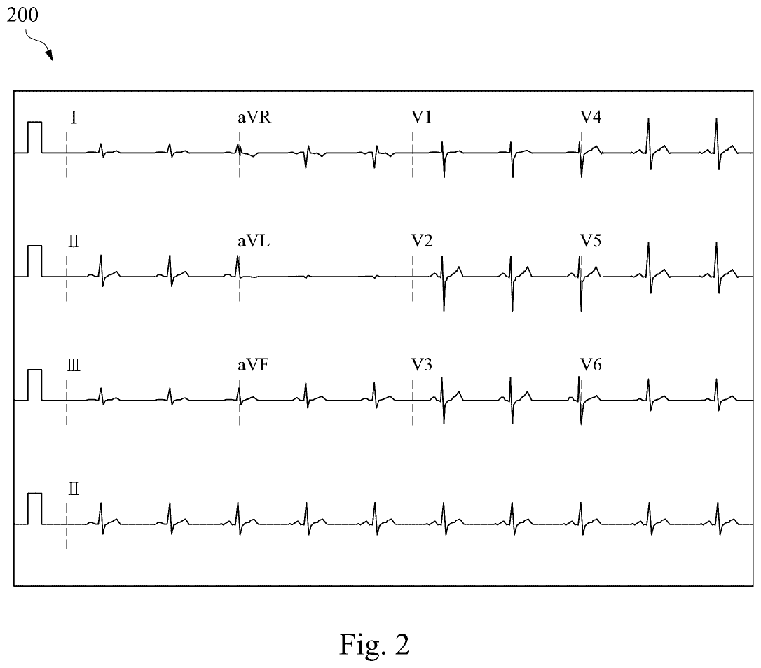

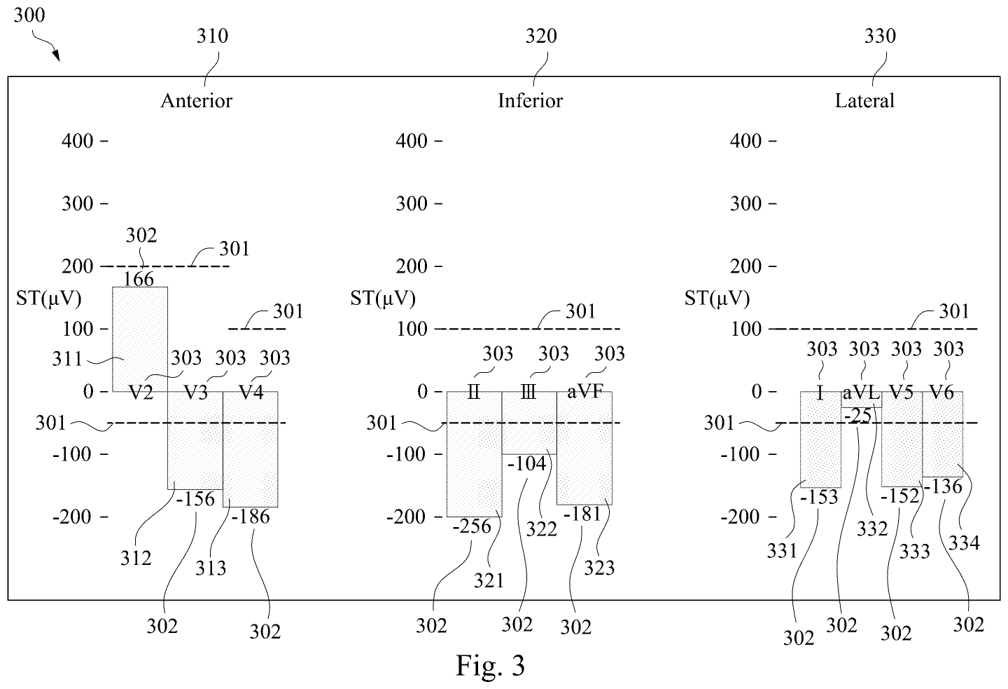

FIG. 1 is a schematic flowchart of an electrocardiogram display method according to an embodiment of the present invention, FIG. 2 is the measured electrocardiogram, and FIG. 3 is a schematic diagram of the electrocardiogram drawn by the electrocardiogram display method of the present invention.

First, referring to FIG. 1, as shown in the figure, the electrocardiogram display method 100 includes the following steps.

First, in step 110, an electrocardiogram measurement device is used to measure a patient's electrocardiogram signal. Generally, referring to FIG. 2, the measured electrocardiogram signal may be drawn as the electrocardiogram 200 in FIG. 2 for medical personnel to determine the patient's heart abnormality.

However, the electrocardiogram display method 100 of the present invention further includes step 120 to calculate the ST amplitudes of the electrocardiogram leads based on the electrocardiogram measurement results.

In addition, in step 130, the electrocardiogram display method 100 further classifies the measured electrocardiogram according to heart regions.

In some embodiments, the aforementioned heart regions can be classified into an anterior heart ECG group 310, an inferior heart ECG group 320, and a lateral heart ECG group 330, to quickly provide medical personnel with a quick electrocardiogram having three classified ECG groups for different orientations and locations of the heart.

Next, in step 150, also referring to FIG. 3, bar charts of the ST amplitudes of a plurality of leads are drawn according to the above classification. In addition, the step of drawing bar charts of the ST amplitudes of the electrocardiogram leads into predetermined distinct groups includes drawing the anterior heart ECG group 310, drawing the inferior heart ECG group 320, and drawing the lateral heart ECG group 330.

In some embodiments, the step of drawing the anterior heart ECG group 310 includes drawing a Lead V2 bar chart 311, drawing a Lead V3 bar chart 312, and drawing a Lead V4 bar chart 313. Preferably, the Lead V2 bar chart 311, the Lead V3 bar chart 312, and the Lead V4 bar chart 313 are drawn adjacent to each other.

In some embodiments, the step of drawing the inferior heart ECG group 320 includes drawing a Lead II bar chart 321, drawing a Lead III bar chart 322, and drawing a Lead aVF bar chart 323. Preferably, the Lead II bar chart 321, the Lead III bar chart 322, and the Lead aVF bar chart 323 are drawn adjacent to each other.

In some embodiments, the step of drawing the lateral heart ECG group 330 includes drawing a Lead I bar chart 331, drawing a Lead aVL bar chart 332, drawing a Lead V5 bar chart 333, and drawing a Lead V6 bar chart 334. Preferably, the Lead I bar chart 331, the Lead aVL bar chart 332, the Lead V5 bar chart 333, and the Lead V6 bar chart 334 are drawn adjacent to each other.

In addition, Lead V1, Lead V2, Lead V3, Lead V4, Lead V5, or Lead V6 are chest leads. Lead I is the potential difference between the left arm (LA) and the right arm (RA), i.e., Lead I=LA −RA. Lead II is the potential difference between the left leg (LL) and the right arm (RA), while Lead III is the potential difference between the left leg (LL) and the left arm (LA). In addition, Lead aVL is an augmented unipolar limb lead for the left upper limb, and Lead aVF is an augmented unipolar limb lead for the left lower limb.

In some embodiments, the anterior heart ECG group 310, the inferior heart ECG group 320, and the lateral heart ECG group 330 are preferably presented on a same medium, for example, a same display screen or a same ECG report page. The anterior heart ECG group 310, the inferior heart ECG group 320, and the lateral heart ECG group 330 are drawn in groups, and the bar charts of leads in the same group are drawn adjacent to each other, and separated from the bar charts of leads in other groups, to highlight the bar charts of the same group of ECGs.

Therefore, medical personnel can quickly understand the abnormal phenomenon occurring in the heart and the heart abnormality location based on the rapid electrocardiogram 300 drawn by the electrocardiogram display method 100 of the present invention.

In some embodiments, in step 140, the electrocardiogram display method 100 of the present invention further includes marking the abnormal threshold values for the electrocardiogram leads on the rapid electrocardiogram 300. Referring to the abnormal threshold line 301 in FIG. 3, corresponding abnormal threshold lines 301 are drawn for different ECG leads. For example, the abnormal threshold lines 301 for the Lead V2 bar chart 311 are −50 microvolts (μV) and +200 microvolts, and the height of the Lead V2 bar chart 311 is 166 microvolts. Therefore, the ST amplitude of the Lead V2 bar chart 311 is normal. Referring further to the Lead V4 bar chart 313 in FIG. 3, the abnormal threshold lines 301 for the Lead V4 bar chart 313 are −50 microvolts (μV) and +100 microvolts, and the height of the Lead V4 bar chart 313 is −186 microvolts. Therefore, the ST segment of the Lead V4 bar chart 313 shows a depression, exceeding the lower limit of the abnormal threshold line 301. Thus, with the information on the rapid electrocardiogram 300, medical personnel may conveniently determine the location of the heart abnormality.

In some embodiments, the abnormal threshold lines 301 can be drawn on the rapid electrocardiogram 300 using solid red lines or dashed lines, to facilitate the interpretation of the electrocardiogram by medical personnel.

In some embodiments, the electrocardiogram display method 100 further includes marking the actual ST amplitude values 302 of the electrocardiogram leads beside the corresponding bar chart, to make the electrocardiogram more convenient for medical personnel to read the abnormal values.

In some embodiments, the electrocardiogram display method 100 further includes drawing the names of the electrocardiogram leads in corresponding lead identification areas 303, e.g. corresponding positions on the horizontal axis, to facilitate medical personnel in confirming the bar charts of the corresponding ECG lead.

In some embodiments, the electrocardiogram display method 100 further includes step 160, comparing the ST amplitudes of the electrocardiogram leads with the abnormal threshold values, and step 170, issuing an alert when the ST amplitude exceeds the abnormal threshold value, for example, by emitting an audio prompt or displaying a different color bar chart on the corresponding bar chart, to remind medical personnel about the abnormal lead and the magnitude of the abnormal value.

Accordingly, the electrocardiogram display method disclosed in the present invention allows medical personnel to easily determine the site of heart damage based on different ECG groups, and the bar charts and abnormal threshold lines make the electrocardiogram convenient to recognize the magnitude of the abnormality. In combination with the actual ST amplitude values marked beside the corresponding bar charts, the electrocardiogram is more convenient for medical personnel to read abnormal values. Furthermore, the lead identification areas marked on the horizontal axis of the bar charts make the electrocardiogram easier for medical personnel to confirm the corresponding ECG leads of the bar charts. Therefore, the rapid electrocardiogram may effectively reduce the time for medical personnel to judge ECG abnormalities in emergencies, thereby improving the efficiency of diagnosis and treatment by medical personnel.

However, the foregoing is only a preferred embodiment of the present invention, and is not intended to limit the scope of implementation of the present invention. Any simple equivalent changes and modifications made according to the scope of the patent application and the description of the invention are still within the scope covered by the present invention. In addition, any embodiment or claim of the present invention does not need to achieve all the objectives, advantages, or features disclosed by the present invention. Furthermore, the abstract and headings are merely for assisting in patent document searches and are not intended to limit the scope of the present invention. Furthermore, terms such as “first” and “second” mentioned in this specification or claims are only used to name elements or distinguish different embodiments or scopes, and are not used to limit the upper or lower limit on the number of elements.

Although the present disclosure has been disclosed above in terms of implementation, it is not intended to limit the present disclosure. Any person with ordinary knowledge in the field may make various variations and modifications without departing from the spirit and scope of the disclosure. Therefore, the spirit and scope of the appended claims should not be limited to the description of the embodiments contained herein.

What is claimed is:

1. An electrocardiogram display method, comprising:
measuring an electrocardiogram to get a measured electrocardiogram;
calculating ST amplitudes of a plurality of electrocardiogram leads according to the measured electrocardiogram;
classifying the measured electrocardiogram according to heart regions; and
drawing bar charts of the ST amplitudes of the electrocardiogram leads into predetermined distinct groups.

2. The electrocardiogram display method as claimed in claim 1, further comprising:
marking abnormal threshold values for the electrocardiogram leads.

3. The electrocardiogram display method as claimed in claim 2, wherein the abnormal threshold values of the electrocardiogram leads are drawn with red abnormal threshold lines.

4. The electrocardiogram display method as claimed in claim 2, further comprising:
comparing the ST amplitudes with corresponding abnormal threshold values of the abnormal threshold values; and
issuing an alert when the ST amplitudes exceed the abnormal threshold values.

5. The electrocardiogram display method as claimed in claim 1, further comprising:
marking actual ST amplitude values beside the bar charts of the ST amplitudes of the electrocardiogram leads.

6. The electrocardiogram display method as claimed in claim 1, further comprising:
drawing names of the electrocardiogram leads in corresponding lead identification areas.

7. The electrocardiogram display method as claimed in claim 1, wherein the step of drawing the bar charts of the ST amplitudes of the electrocardiogram leads into predetermined distinct groups comprises:
drawing an anterior heart ECG group;
drawing an inferior heart ECG group; and
drawing a lateral heart ECG group.

8. The electrocardiogram display method as claimed in claim 7, wherein the step of drawing the anterior heart ECG group comprises:
drawing a Lead V2 bar chart;
drawing a Lead V3 bar chart; and
drawing a Lead V4 bar chart.

9. The electrocardiogram display method as claimed in claim 8, wherein the step of drawing the inferior heart ECG group comprises:
drawing a Lead II bar chart;
drawing a Lead III bar chart; and
drawing a Lead aVF bar chart.

10. The electrocardiogram display method as claimed in claim 9, wherein the step of drawing the lateral heart ECG group comprises:
drawing a Lead I bar chart;
drawing a Lead aVL bar chart;
drawing a Lead V5 bar chart; and
drawing a Lead V6 bar chart.

11. An electrocardiogram display method, comprising:
measuring an electrocardiogram to get a measured electrocardiogram;
calculating ST amplitudes of a plurality of electrocardiogram leads according to the measured electrocardiogram;
classifying the measured electrocardiogram according to heart regions; and
drawing bar charts of the ST amplitudes of the electrocardiogram leads into an anterior heart ECG group, an inferior heart ECG group and a lateral heart ECG group.

12. The electrocardiogram display method as claimed in claim 11, further comprising:
marking abnormal threshold values for the electrocardiogram leads.

13. The electrocardiogram display method as claimed in claim 12, wherein the abnormal threshold values of the electrocardiogram leads are drawn with red abnormal threshold lines.

14. The electrocardiogram display method as claimed in claim 12, further comprising:
comparing the ST amplitudes with corresponding abnormal threshold values of the abnormal threshold values; and
issuing an alert when the ST amplitudes exceed the abnormal threshold values.

15. The electrocardiogram display method as claimed in claim 11, further comprising:
marking actual ST amplitude values beside the bar charts of the ST amplitudes of the electrocardiogram leads.

16. The electrocardiogram display method as claimed in claim 11, further comprising:
drawing names of the electrocardiogram leads in corresponding lead identification areas.

17. The electrocardiogram display method as claimed in claim 11, wherein the anterior heart ECG group comprises a Lead V2 bar chart, a Lead V3 bar chart and a Lead V4 bar chart.

18. The electrocardiogram display method as claimed in claim 17, wherein the inferior heart ECG group comprises a Lead II bar chart, a Lead III bar chart and a Lead aVF bar chart.

19. The electrocardiogram display method as claimed in claim 18, wherein the lateral heart ECG group comprises a Lead I bar chart, a Lead aVL bar chart, a Lead V5 bar chart and a Lead V6 bar chart.
